(19) United States
(12) Patent Application Publication
(10) Pub. No.: US 2009/0038752 A1
(43) Pub. Date: Feb. 12, 2009

(54) REINFORCED BALLOON FOR A CATHETER

(76) Inventors: Adel Weng, San Diego, CA (US); William M. Appling, Granville, NY (US)

Correspondence Address: PATENT DOCKET CLERK;COWAN, LIEBOWITZ & LATMAN, P.C., 1133 AVENUE OF THE AMERICAS, NEW YORK, NY, 10036, US

(21) Appl. No.: 11/658,391

(22) PCT Filed: Feb. 8, 2006
(86) PCT No.: PCT/US2006/004511

§ 371 Date: Oct. 16, 2008

Related U.S. Application Data
(60) Provisional application No. 60/651,696, filed on Feb. 9, 2005.

(51) Int. Cl. A61M 25/10; B32B 37/06; C09J 5/06

(52) U.S. Cl. 156/276; 604/103.09; 156/311

(57) ABSTRACT

An inflatable balloon, and method of making same, for a medical catheter includes a base layer or non-compliant balloon substrate, a reinforcing layer, an adhesive. layer adhering the reinforcing layer to the non-compliant balloon substrate, and a top coat layer. The reinforcing layer is a single ply matrix of interwoven strands applied to the non-compliant balloon substrate. There are preferably three sets of strands in the single ply matrix. One set of strands extends in a longitudinal direction. Another set of strands extends circumferentially in a helical fashion with a clockwise orientation at an angle of approximately 65° with the strands that extend in the longitudinal direction. The third set of strands extends circumferentially in a helical fashion with a counter-clockwise orientation at an angle of approximately 65° with the strands that extend in the longitudinal direction. The three sets of strands are interwoven with one another by a braiding machine so as to provide a single ply of interwoven strands to achieve a uniform, reproducible reinforcing matrix.

### CROSS REFERENCE TO RELATED APPLICATIONS

This application claims priority to U.S. Provisional Application No. 60/651,696, filed Feb. 9, 2005, which is hereby incorporated herein by reference.

### FIELD OF THE INVENTION

The present invention relates to a reinforced high strength balloon adapted for use on a catheter and more particularly adapted for use on a percutaneous transluminal angioplasty catheter.

### BACKGROUND OF THE INVENTION

Treatment of stenosis by angioplasty balloon catheters is well known. Typically a lesion is opened by inflating a balloon catheter at moderate inflation pressures up to 18-20 atms. Some stenotic lesions can be highly resistant to opening at these pressures and occasionally standard balloon catheters are not strong enough to sufficiently open such lesions. For example, when treating a stenosis that occurs at the venous anastomosis of a dialysis graft, it is found that often these lesions may require pressure in excess of 30 atmospheres to sufficiently open the stenosis. To address the need for balloons with higher pressure ratings, high-strength, reinforced balloons were developed. These balloons are capable of withstanding pressures in excess of 30 atmospheres and are able to open resistant lesions.

A number of patents have been issued which cover the concept of a reinforced balloon catheter. Several of these patents are directed to compliant balloon catheters with reinforcing members that restrict expansion of the compliant balloon upon inflation to a predetermined diameter. For example, U.S. Pat. No. 4,706,670 to Andersen et al describes a compliant dilation balloon catheter having a filament reinforced shaft and balloon. When the balloon is expanded, the filament angles change to align with a critical angle to prevent further expansion of the balloon. The reinforcement also prevents foreshortening in the balloon length upon expansion. Other patents covering reinforcing members to control overexpansion and foreshortening are U.S. Pat. No. 5,201,706 and U.S. Pat. No. 5,330,429 to Noguchi et al, U.S. Pat. No. 5,112,304 to Barlow et al and U.S. Pat. No. 5,647,848 to Jorgensen. The reinforced material serves to control the inflated diameter and length of a non-compliant balloon rather than to increase pressure capabilities.

A number of patents have also issued covering the use of reinforcement elements on non-compliant balloons. One such patent is U.S. Pat. No. 6,629,952 to Chien et al. This patent discloses a high pressure balloon catheter wherein the inner and outer shafts are reinforced with a braided ribbon member. The reinforcement provides strength against rupture under pressure and prevention of kinking and advancement through tortuous vasculature. In one embodiment the braided member extends from the outer shaft over the balloon. In another embodiment, a separate braided structure extends over the balloon. Although Chien et al discloses longitudinal reinforcement elements, these elements are restricted to the catheter shaft and function to minimize kinking.

In U.S. Pat. No. 6,746,425, Beckham describes a non-compliant angioplasty balloon with two separate distinct fiber layers each consisting of a high-strength inelastic fibers. The first fiber layer is positioned along the entire length of the longitudinal axis of the balloon with all its fibers extending in the longitudinal direction. The fibers of the second layer are wound radially and extend in a direction substantially perpendicular to the fibers of the first layer. This design provides both radial and longitudinal reinforcement producing a high-pressure balloon capable of withstanding pressures up to 30 atms without rupture.

The method of manufacturing this balloon is time-consuming and requires separate steps to place the first and second fiber layers. The first longitudinal fiber layer is particularly time-consuming because it requires the precise placement of up to 30 individual fibers on the balloon base. The second layer and optional third layer application involves circumferentially winding the fiber with up to 54 wraps per inch. This manufacturing technique may result in misalignment of individual longitudinal fibers prior to the application of the polyurethane coating layers. It is important that the reinforced balloon maintain a small overall deflated profile with a minimum wall thickness to allow ease of insertion and advancement of the catheter through tortuous anatomy.

Reinforcing strands are known as shown, for example, in U.S. Pat. No. 6,156,254 to Andrews.

### SUMMARY

One aspect of this invention is a reinforced balloon for an angioplasty catheter in which the reinforcing layer is composed of three sets of strands interwoven with each other by a machine to produce a single layer of interwoven strands.

A related aspect of this invention, which is achieved by the machine fabrication, is a reinforced balloon in which the reinforcing layer has its strands uniformly deployed and in which reinforcement characteristics are consistent from balloon to balloon.

A further aspect of this invention, also achieved by machine fabrication, is providing a reinforced balloon by a relatively rapid and low cost process.

Yet a further aspect of this invention is to provide a balloon having a design which increases the burst strength of such balloons as contrasted with prior balloons. That is, to provide a balloon which, for a given wall thickness, provides increased burst strength than that provided by previously known balloons.

A further related aspect of the invention is a balloon design that has a minimal wall thickness.

Another aspect of the invention is making an inflatable balloon for a medical catheter by applying a reinforcing ply having a first plurality of strands extending in a first direction, and a second plurality of strands extending in a second direction which is non-parallel with said first direction to a base ply, thus forming a combination of base ply and reinforcing ply, in which the strands of the second plurality are interwoven with the stands of the first plurality, and in which the reinforcing ply is layered with the base ply.

### BRIEF DESCRIPTION

The objects of this invention are achieved by a single ply matrix of interwoven strands applied to a non-compliant balloon substrate as a single layer. There are preferably three sets of strands in the single layer.

One set of strands extend in a longitudinal direction.

Another set of strands extend circumferentially in a helical fashion with a clockwise orientation at an angle of approximately 65° with the strands that extend in the longitudinal direction.

Another set of strands extend circumferentially in a helical fashion with a counter-clockwise orientation at an angle of approximately 65° with the strands that extend in the longitudinal direction.

These three sets of strands are interwoven with one another by a braiding machine so as to provide a single ply of interwoven strands to achieve a uniform, reproducible reinforcing matrix.

The result is an enhanced reinforced non-compliant balloon.

### BRIEF DESCRIPTION OF THE DRAWINGS

FIG. 1 is an elevation view, in somewhat schematic form, showing the three interwoven sets of strands (22), (24), (26) which constitute the reinforcing ply for the high pressure balloon. In FIG. 1, certain of the longitudinal strands are deleted to facilitate presentation and simplify the illustration.

FIG. 1A is a larger scale view of a portion of the balloon of FIG. 1, showing in greater detail the relationship between individual strands of each of the three sets of strands.

FIG. 2 is a longitudinal sectional view through the wall of the balloon showing the relationship between one longitudinal strand (22a) and the circumferential strands, (24), (26).

FIG. 3 is a flow chart showing the steps employed in fabricating the reinforced balloon of FIG. 1.

FIG. 4 is a longitudinal section view through the wall of the balloon after a curing step in the process of making the balloon.

### DETAILED DESCRIPTION OF THE INVENTION

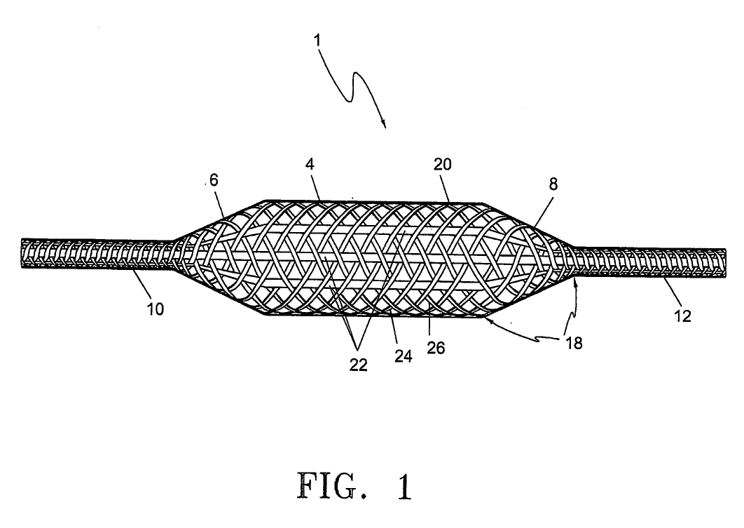

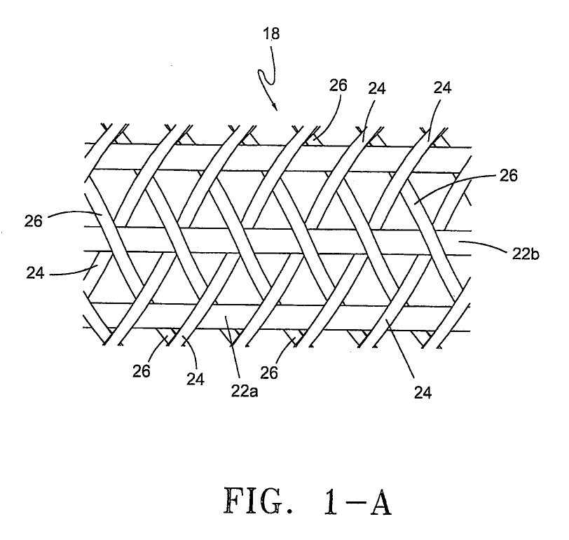

Referring to FIG. 1, a plan view of the reinforced balloon 1 is shown illustrating the interwoven multi-strand reinforcing ply or layer. The reinforced balloon 1 is comprised of proximal and distal balloon neck portions (10) and (12) respectively, proximal and distal balloon cone portions (6) and (8) respectively, and a balloon body portion (4). The distal and proximal balloon neck portions (10) and (12) are bonded to a catheter shaft (not shown) using bonding techniques commonly known in the art. The proximal and distal cone portions (6) and (8) gradually increase in diameter from the neck portions 10/12 diameter to the body portion (4) diameter. The balloon body portion (4) is designed to contact the vessel wall and when inflated is of a constant diameter.

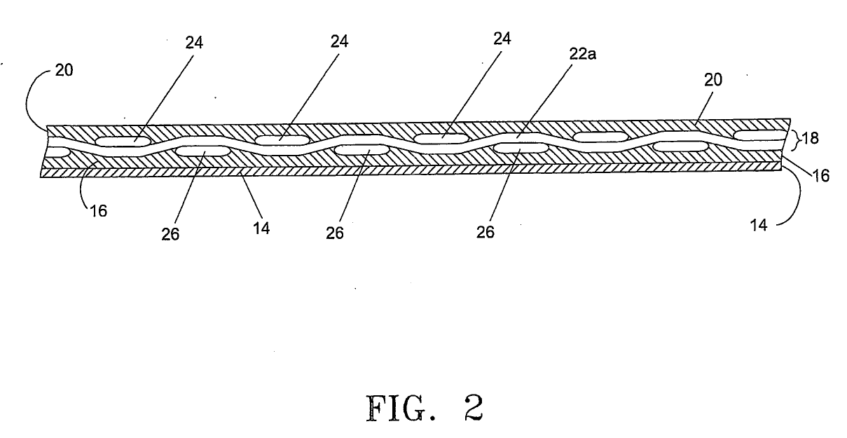

FIG. 1 illustrates the reinforcing ply (18), which is one of the four plies of material that comprise the laminate, reinforced balloon (1). This fiber ply (18) is applied directly to a base PET ply (14) (shown in FIG. 2) to which adhesive has been applied. The reinforcing ply (18) is comprised of a set of strands (22) extending in a longitudinal direction and two sets of circumferential strands (24) and (26), each of which are arranged helically with respect to the longitudinal axis of the balloon (1). These three sets of strands are interwoven together using a known braiding machine to produce the single ply (18) that has improved strength and abrasion-resistance properties. A top ply (20) of polyurethane is then applied over the reinforcing strand ply (18).

FIG. 1 illustrates a preferred interwoven strand pattern. Strands are identified in terms of the angle of their placement on the balloon base PET ply (14) relative to the longitudinal axis of the balloon. A strand placed parallel to the longitudinal axis is defined herein as having a relative zero angle. Helically placed strands are oriented at an angle of between 30-70 degrees relative to the longitudinal axis.

As shown in FIG. 1, the strand pattern consists of multiple longitudinal strands (22) captured between two sets of helically interwoven strands (24) and (26) running in clockwise and counter-clockwise directions around the balloon base ply (14) oriented at a preferred angle of 60 to 70 degrees.

In the preferred pattern, the strand sets (24) and (26) are interwoven with each other. An example of interwoven strands is such that strand (24) crosses in a repeating pattern that proceeds under two strands (26) and then over two strands (26). Cross-over and cross-under points where two strands intersect is defined herein as a pick. The number of picks per inch is the number of interwoven contact points within an inch and represents the density of the strand pattern.

Other interwoven patterns are also within the scope of this invention. For example, the helical stands (24) may cross over one strand (26) and then under one strand (26) rather than the “over-two, under-two” pattern. Alternatively, two strands (24) may be woven in parallel as a single strand using either cross-over pattern described above to produce a more complete coverage of the balloon surface. Other braiding or interwoven patterns are also contemplated herein.

The helical strands (24) and (26) provide increased hoop strength to the balloon. The density of the strand pattern may be modified by varying the number of strands (24) and (26) used in the weaving process, as well as varying the speed of the braider and also by the denier size of the fiber strands. A more dense strand pattern will produce a stronger balloon. Preferably, the strand pattern will be dense enough to limit the open, un-reinforced space between the strands to less than 1.0 mm².

The longitudinal strands (22) are interwoven with the helical strands (24) and (26) such that a particular longitudinal strand (22a) passes under strands (24) and over strands (26) in a repeating pattern for the length of the balloon. The next longitudinal strand (22b) passes over the strands (24) and under the strands (26). Alternative patterns are also within the scope of this invention.

The number of longitudinal strands (22) woven over the balloon may vary but is preferably sixteen with a range of four to thirty-two. The actual preferred number of longitudinal strands will depend primarily on the balloon diameter size. In general, the number of longitudinal strands will be half the total number of helical strands. The longitudinal strands provide the balloon (1) with increased longitudinal strength as well as preventing failures at inflated pressures.

The combined interwoven longitudinal and helically oriented strands produce a single reinforcement ply with three sets of interwoven reinforcing strands that provides a balloon with optimal reinforcement to prevent both circumferential and longitudinal bursts. In addition, the use of interwoven methods to create a single ply of interwoven strands rather than a plurality of strand layers produces a balloon that has a thin wall thickness and can be manufactured at a low cost due to the automated process for applying the strands. Because the interwoven configuration results in a tubular ply with adjacent strands supporting each other, thinner strands can be used without compromising strength.

Referring to FIG. 2, the four plies of the reinforced balloon 1 of the current invention are shown prior to curing or otherwise baking under pressure. A longitudinal cross-section of a balloon wall segment depicting the four plies that form the balloon structure is shown. Prior to the final step of curing, in the process of forming the reinforced balloon, the four plies are layered as depicted in FIG. 2. During the final curing or baking step these four balloon plies are compressed together into a united laminate structure having enhanced strength properties. Thus, after curing the four plies form a composite single united laminate structure. As used herein, the expression “laminate structure” refers to the composite single united laminate structure created after curing.

Balloon (1) is comprised of an inner polyethylene terephthalate (PET) blown balloon base ply (14), an adhesive coating ply (16), the reinforcing multi-strand ply (18) and the polyurethane outer ply (20). The adhesive coating ply (16) adheres to the reinforcing ply (18) and fills in the spaces between strands. Similarly, the polyurethane top ply (20) infuses between the strands filling the voids between the strands and thus encapsulating the strands.

The inner PET balloon base ply (14) is formed using conventional extrusion and balloon blowing methods commonly known in the art. The extruded noncompliant PET tubing is blown into an expanded balloon shape using a cavity mold on a balloon blowing machine. Temperature, pressure and axial stretch parameters are used to produce a very thin balloon base structure (14) with minimal shrinkage upon which the reinforcement ply (18) will be applied. As an example, an 8 mm PET balloon structure will have a double wall thickness of between 0.4 and 0.8 mil (0.004 to 0.008 inch).

Although PET is the preferred material for the base balloon, other non-compliant materials may be used. These materials include high-strength, polymers such as polyamides, polyamide copolymers, PET copolymers, high durometer or engineering thermoplastic elastomers, blends and alloys of the above.

The adhesive coating ply (16) is next applied to the inflated base PET balloon ply (14). The adhesive (16) is preferably a two-part polyurethane adhesive that is applied uniformly as a thin coating to the outer surface of the balloon ply (14) using application techniques commonly known in the art such as a wiping or brush-on technique. The adhesive should exhibit a relatively low viscosity to allow uniform application across the entire surface of the ply (14). The adhesive is then allowed to partially, but not completely, cure to achieve a level of tackiness sufficient to cause the reinforcing strand ply (18) to adhere to the ply (14). A polyurethane adhesive is preferred to provide optimal bonding with the outer ply (20). Other one and two-part adhesives or sprayable non-polyurethane adhesives may also be used.

The reinforcing strand ply (18) is a key aspect of this invention. Unlike prior art reinforced balloons in which multiple layers of fibers or strands are sequentially applied to the adhesive coated base balloon structure, the interwoven strand ply (18) is laid down directly as a single ply over the inflated balloon using a modified braider machine. Because the inter-weaving process is automated as described in more detail below, it is advantageous over prior art designs which require the manual application of individually cut longitudinal strands followed by either one or two circumferential or helical winding steps. Also, the automation of the strand application function provides a much higher degree of consistency in final pattern arrangement than in prior art designs.

The strands may be any type of high-strength noncompliant material such as high-tenacity para-aramid or thermotropic liquid crystal polyester-polyarylate. These materials produce strands that are up to eight times as strong as steel and up to three times as strong as fiberglass, polyester and nylon of the same weight. Other non-elastic, high-strength materials may also be used. These materials may include ultra-high molecular weight polyethylene or extended chain polyethylene, poly-p-phenylene-2,6-benzobisoxazole and poly-paraphenylene terephthalamide.

The size of the strand is variable but preferably between 25 and 200 denier. Higher denier strands yield higher burst strengths to the balloon but have the drawback of increasing the thickness of the balloon. A combination of denier sizes may be utilized to maximize strength characteristics while minimizing wall thickness of the finished balloon. For example, the longitudinal fibers may be of a different material and denier than the helical strands.

The strand material is comprised of individual fibers or yarns that are generally round in shape. Interweaving the multi-fiber strands with appropriate tension as well as the pressure during the curing step causes the individual fibers within the strand to spread out across the surface of the balloon, resulting in a flattened profile of the strands.

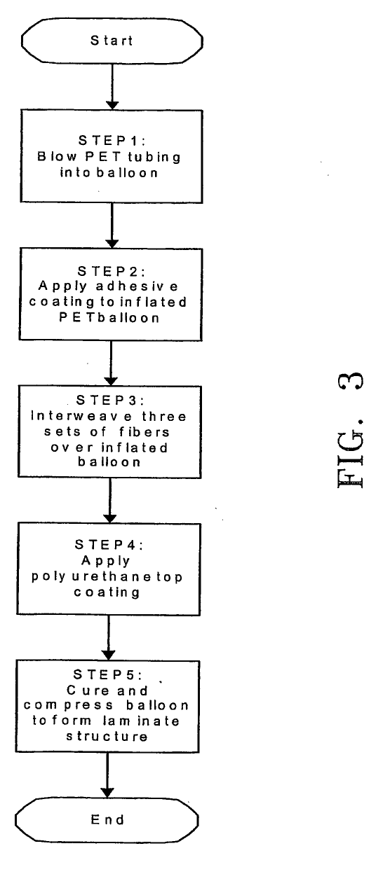

The method of manufacture is described with reference to FIG. 3, which illustrates the individual processing steps of forming the balloon laminate structure. As previously described, the base PET balloon structure (14) is first producing using conventional balloon blowing techniques. The first adhesive ply (16) is then applied to the base balloon (14) using brush or wipe on application techniques.

Step 3 is the inter-weaving. To produce the desired strand pattern, a modified braiding machine can be used. Typically 32 circumferential fiber carriers are loaded into the braider. Longitudinal fiber carriers are stationary and may number between four and sixteen for an 8 mm balloon. The inflated balloon substrate, mounted on a cannula, is placed into the braider machine and is moved vertically at a fixed or variable speed while the strands are applied. Strand density may be varied by varying the total number of carriers used, the vertical speed at which the balloon is moved through the braider and the size of the individual strand. These parameters may be adjusted to minimize the open, un-reinforced space between the strands.

As the balloon substrate is moved vertically through the strand application area, the inter-weaving strand pattern is applied sequentially to the distal neck (12) of the balloon, the distal cone (8) section, the body (4), the proximal cone (6) section, and the proximal neck (10) of the balloon. The picks per inch of each balloon section will differ slightly with the most dense pattern being on the neck (10) section because of its reduced surface area The density will decrease as the machine application of strands moves from the neck (10) to the cone and on to the body (4) section where it will be the least dense.

The slightly denser pattern on the cone area is advantageous in that these sections are more vulnerable to rupture or damage from advancement or withdrawal of the catheter during the medical procedure. The strand pattern on necks (10) and (12) reinforces the bond area where the balloon attaches to a shaft. Providing additional reinforcement to these sections of the balloon decreases the likelihood of balloon failure at the cone section.

After the strand ply has been applied to the base PET balloon, an aqueous polyurethane solution is sprayed over the inflated balloon to form the top coating layer (20). This process is represented by Step 4 in FIG. 3. Because of its liquid form, the top coating ply (20) infuses between the strands providing a barrier to abrasion. The ply (20) is preferably of polyurethane based solution. During the final baking step, explained in more detailed below, the backbone polyurethane polymer in the aqueous spray solution will soften, flow and infuse between the strands to join with adhesive ply (16) to form a laminate structure with superior strength properties. Other top coat layers are within the scope of this invention including adhesive film, non-adhesive materials such as PET formed into an outer balloon which when heated and cured forms the final laminate structure.

Although an aqueous polyurethane solution is preferred because of its ease of use and non-toxic qualities, other water-based and solvent-based polyurethane coatings may also be used to form the top ply (20). The coating may also be applied using a brush-on or dipping technique.

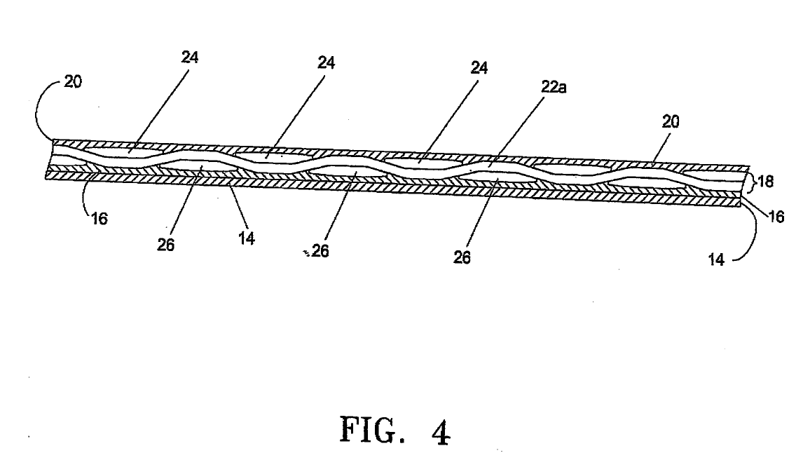

As a final step in the manufacture of the reinforced balloon, the four ply balloon structure is heat or pressure cured to produce the single composite united laminate structure as shown in FIG. 4. FIG. 4 illustrates the final thin laminate structure in which the matrix has flattened and spread out over the base balloon ply and in which the top coat and base coat have been fused together to form the thin high strength balloon laminate structure. One method of performing this curing step is with the use of a heated curing mold. The balloon structure prior to curing is inserted into a heated chamber and the walls are compressed. The purpose of this step is to fuse and compress the individual plies of the balloon into a thin laminate structure. The application of heat and pressure to the balloon serves several purposes.

The baking under pressure process causes the polyurethane polymer of the top ply (20) to infuse between the strands (22), (24), (26) and bond with the polyurethane adhesive ply (16) creating a stronger structure. The internal pressure in the mold which may go as high as 250 psi causes the strands to further flatten across the surface of the balloon. This results in more balloon structure area being reinforced. As shown in FIG. 4, it also reduces the cross-sectional thickness of the final balloon and provides enhanced abrasion resistance.

The curing process shown in Step 5 of FIG. 3 may be accomplished using an air baking chamber. The balloon structure is inflated within the heated air chamber to a pressure that exceeds the pressure of the chamber. The higher pressure within the balloon structure, combined with the elevated temperatures of the chamber, causes compression of the balloon structure with a corresponding decrease in wall thickness. The air baking curing step is advantageous in that a more consistent uniform pressure is applied to the entire balloon surface area. In addition, since the balloon surface is not in contact with a mold structure, the fiber matrix is not disturbed during the insertion or removal of the balloon from the chamber.

A major advantage of the deployment of interwoven strands as the reinforcement layer is the ability to use standard, known machines (often called braiding machines) to lay down the strands in an interwoven fashion. The machine may have to be modified in a fashion obvious in the art to insert the longitudinal strands into the weaving of the two sets of circumferentially woven strands. The adaptability of the three way interwoven matrix of strands to machine fabrication substantially increases the speed of fabrication and decreases the cost of the balloons. It also provides a much more uniform balloon in which the spacing between adjacent strands within a set of strands is uniform.

One further result, due to the uniform spacing, is that for a given strand density, the reinforcement strength is uniform. This contributes to a balloon which, for a given wall thickness, has enhanced burst strength.

In addition, the inter-weaving of the strands provides a single layer matrix of interwoven strands.

It is believed that the interwoven relationship between the strands aids in bringing about a uniform distribution of the forces that are resolved by the network of strands. This further contributes to a balloon which, for a given wall thickness, has enhanced burst strength.

It should be understood that an individual strand can be composed of multiple individual fiber elements or can be a single melt spun element.

One of the advantages of having a multi-fiber strand is that the fibers tend to spread out causing the strand to become flattened during the process of applying the strand to the balloon and during the process of compressing the balloon sidewall to assure a minimum thickness balloon. The number of individual fibers will depend upon the denier of the strand. This serves to maintain the thin wall characteristic of the balloon and also to provide a greater area of reinforcement of the balloon.

For example, in a 25 denier strand, there may be five individual fibers, in a 55 denier strand, there may be twenty individual fibers and in a 100 denier strand, there may be twenty-five individual fibers.

The term “strand” is used herein to refer to the multiple fiber strand. The term “fiber” will be used herein to refer to the individual fibers that constitute the strand. But strands having multiple fibers are preferred because they permit the strand to flatten out during the fabrication process and thus contribute to maintaining a thin sidewall.

In one embodiment involving a balloon that is 40 mm long, (excluding the 10 mm cones at the ends of the balloon) and has an 8 mm inflated diameter, the circumferential strand deployment is as follows. Each strand is at an angle of 65° to the longitudinal strands and makes approximately 2½ rotations (1,000 degrees) on each inch (2.54 cm) of balloon body length. This 40 mm balloon is approximately 1.57 inches; so that the leading strand will make approximately four rotations over the main body of the balloon.

While certain novel features of this invention have been shown and described above, the present invention may be embodied in other specific forms without departing from the spirit or essential characteristics of the invention such as materials, braiding configurations and process steps. The described embodiments are to be considered in all respects only as illustrative and not as restrictive.

Various omissions, modifications, substitutions and changes in the forms and details of the device illustrated and in its operation can be made by those skilled in the art without departing in any way from the spirit of the present invention.

1. An inflatable balloon for a medical catheter comprising:
a noncompliant combination of a base ply and a reinforcing ply;
said reinforcing ply comprising a first plurality of strands extending in a first direction, and a second plurality of strands extending in a second direction which is non-parallel with said first direction;
said strands of said second plurality being interwoven with the stands of said first plurality; and
said reinforcing ply being layered with said base ply to form a layered structure.

2. The inflatable balloon of claim 1 wherein said base ply of said noncompliant combination is noncompliant.

3. The inflatable balloon of claim 1 wherein said reinforcing ply of said noncompliant combination is noncompliant.

4. The inflatable balloon of claim 1 wherein said base ply and said reinforcing ply of said noncompliant combination are noncompliant.

5. The inflatable balloon of claim 1 further comprising an adhesive ply adhering said reinforcing ply with said base ply.

6. The inflatable balloon of claim 5 further comprising a top coat ply over said reinforcing ply.

7. The inflatable balloon of claim 6 wherein said base ply, said reinforcing ply, said adhesive ply and said top coat ply together form a composite laminate structure.

8. The inflatable balloon of claim 1 wherein said strands of said reinforcing ply are chosen from among high tenacity para-aramid or thermotropic liquid crystal polyester-polyacrylate.

9. The inflatable balloon of claim 1 wherein said strands of said reinforcing ply are comprised of a plurality of individual fibers.

10. The inflatable balloon of claim 1 wherein said balloon has a distal neck portion, a proximal neck portion, a body portion therebetween, a longitudinal axis, a longitudinal length defined by the distance from the distal end of the distal neck to the proximal end of the proximal neck, said longitudinal length being larger than the outside diameter of said body portion.

11. The inflatable balloon of claim 10 wherein said reinforcing ply extends over the entire area of said distal neck portion, said proximal neck portion and said body portion.

12. The inflatable balloon of claim 10 wherein said first and said second directions each form an angle of between 30° and 70° relative to said longitudinal axis.

13. The inflatable balloon of claim 12 wherein said first and said second directions each form an angle of between 60° and 70° relative to said longitudinal axis.

14. The inflatable balloon of claim 13 wherein said first and said second directions each form an angle of approximately 65° relative to said longitudinal axis.

15. The inflatable balloon of claim 10 wherein said first direction is substantially parallel to said longitudinal axis, and wherein said second direction forms an angle of between 30° and 70° relative to said longitudinal axis.

16. The inflatable balloon of claim 15 wherein said second direction forms an angle of between 60° and 70° relative to said longitudinal axis.

17. The inflatable balloon according to claim 15 further comprising a third plurality of strands, the strands of said third plurality of strands being interwoven with the strands of said first and second plurality of strands and extending in a third direction forming an angle of between 60° and 70° with said longitudinal axis.

18. The inflatable balloon according to claim 16, wherein said second plurality of strands are oriented in a positive direction relative to said longitudinal axis, and wherein said third plurality of strands are oriented in a negative direction relative to said longitudinal axis.

19. A reinforced balloon on a catheter, the balloon having a longitudinal axis, comprising: a noncompliant combination of a substrate, and a reinforcing layer of interwoven strands adhered to said substrate, said interwoven strands including first, second and third sets of strands, said first set of strands being a set of longitudinal strands oriented in a direction substantially parallel to said longitudinal axis, said second set of strands being a set of strands oriented in a positive direction relative to said longitudinal axis, said third set of strands being a set of strands oriented in a negative direction relative to said longitudinal axis, wherein individual strands of each of said first, second and third sets of strands are interwoven with strands of each of the other sets of strands.

20. The reinforced balloon of claim 19 wherein each strand of said second set is oriented at a clockwise angle of approximately between +30 and +70 degrees to said set of longitudinal strands and each strand of said third set is oriented at a counter-clockwise angle of approximately between −30 and −70 degrees to said set of longitudinal strands.

21. The reinforced balloon of claim 20 wherein said angle of said strands is approximately between 60 and 70 degrees.

22. The reinforced balloon of claim 21 wherein said angle of said strands is approximately 65 degrees.

23. The reinforced balloon of claim 19 wherein at least some of said strands are composed of multiple fiber elements.

24. The reinforced balloon of claim 19 wherein said substrate is non-compliant.

25. The reinforced balloon of claim 19 wherein each of said sets of strands comprises multiple strands.

26. The reinforced balloon of claim 19 further comprising a top coat layer covering said reinforcing layer.

27. A method of making an inflatable balloon for a medical catheter comprising:
applying a reinforcing ply having a first plurality of strands extending in a first direction, and a second plurality of strands extending in a second direction which is non-parallel with said first direction to a base ply forming a combination of base ply and reinforcing ply;
wherein said strands of said second plurality being interwoven with the stands of said first plurality, and said reinforcing ply being layered with said base ply to form a laminate structure.

28. The method of claim 27 further comprising applying an adhesive to said base ply prior to applying said reinforcing ply.

29. The method of claim 27 further comprising applying a top coat ply over said reinforcing ply after applying said reinforcing ply.

30. The method of claim 29 further comprising curing said base ply, said reinforcing ply, said adhesive ply and said top coat ply into said laminate structure.

31. The method of claim 27 wherein said balloon has a distal neck portion, a proximal neck portion, a body portion therebetween, a longitudinal axis, a longitudinal length defined by the distance from the distal end of the distal neck to the proximal end of the proximal neck, said longitudinal length being larger than the outside diameter of said body portion.

32. The method of claim 31 wherein said reinforcing ply is applied over the entire area of said distal neck portion, said proximal neck portion and said body portion.

33. The method of claim 27 wherein said reinforcing ply comprises a third plurality of strands, the strands of said third plurality of strands being interwoven with the strands of said first and second plurality of strands and extending in a third direction forming an angle of between 60° and 70° with said longitudinal axis.

34. The method of claim 27 wherein said second plurality of strands of said reinforcing ply are oriented in a positive direction relative to said longitudinal axis, and wherein said third plurality of strands of said reinforcing ply are oriented in a negative direction relative to said longitudinal axis.

35. The method of claim 27 wherein said strands of said reinforcing ply are comprised of a plurality of individual fibers.

36. The method of claim 27 wherein said strands of said reinforcing ply are chosen from among high tenacity para-aramid or thermotropic liquid crystal polyester-polyacrylate.

37. The method of claim 27 wherein said base ply of said combination is noncompliant.

38. The method of claim 27 wherein said reinforcing ply of said combination is noncompliant.

39. The method of claim 26 wherein said base ply and said reinforcing ply of said combination are noncompliant.

40. A method of making an inflatable balloon for a medical catheter comprising:
applying a reinforcing ply to a base ply forming a layered combination of base ply and reinforcing ply, said reinforcing ply having a first plurality of strands extending in a first direction, and a second plurality of strands extending in a second direction which is non-parallel with said first direction, said strands of said second plurality being interwoven with the stands of said first plurality,
applying a top coat ply over said reinforcing ply after said reinforcing ply is applied to said base ply; and
curing said layered combination of base ply and reinforcing ply with said top coat ply thereby forming a laminate structure.

41. The method of claim 40 further comprising applying an adhesive ply to said base ply prior to applying said reinforcing ply.

42. The method of claim 40 wherein said curing is carried out by inserting said layered combination of base ply and reinforcing ply with said top coat into a heated curing mold, the walls of which are compressed into contact with said top coat to form said laminate structure.

43. The method of claim 40 wherein said curing is carried out by inserting said layered combination of base ply and reinforcing ply with said top coat into an air baking chamber, inflating said layered combination of base ply and reinforcing ply so that the pressure within said layered combination exceeds the pressure within said air baking chamber, and heating the air within said chamber thereby forming said laminate structure.
